(19) United States
(12) Patent Application Publication
(10) Pub. No.: US 2012/0316384 A1
(43) Pub. Date: Dec. 13, 2012

(54) METHOD FOR TREATMENT OF PELVIC ORGAN PROLAPSE CONDITIONS

(76) Inventor: Kelly B. Arnold, Sparta, NJ (US)

(21) Appl. No.: 13/157,564

(22) Filed: Jun. 10, 2011

(51) Int. Cl. A61F 2/02

(52) U.S. Cl. 600/30

(57) ABSTRACT

A method for treating pelvic organ prolapse conditions via a vaginal approach, including identifying the presumptive apex of the vagina and the ischial spine; pressing the presumptive apex of the vagina onto the ischial spine; while maintaining contact between the presumptive apex of the vagina and the sacrospinous ligament, continuously sweeping the presumptive apex of the vagina in a dorso-medial direction from the ischial spine along the sacrospinous ligament to a first location approximately two centimeters from the ischial spine; and while maintaining contact between the presumptive apex of the vagina and the sacrospinous ligament, attaching the presumptive apex to the sacrospinous ligament at the first location using a first tissue anchor.

### FIELD OF THE INVENTION

The present invention relates generally to the field of surgical procedures, and more particularly to methods for treating pelvic organ prolapse conditions.

### BACKGROUND

Pelvic organ prolapsed conditions arise when the normal structural support of the vagina is lost, resulting in the descending or dropping of the bladder, urethra, uterus, cervix or rectum within the pelvic cavity. As the organs descend into the vaginal space, bulging tissues can sometimes be felt through the vaginal opening. This condition is unpleasant and can have life altering consequences.

One method for treating these types of disorders involves securing the apex of the vagina to the sacrospinous ligament or other structurally sound tissue within the pelvic cavity. In many circumstances, a component of anterior and posterior prolapse is directly the result of apical support defects. By providing support to the vaginal apex, any apically related prolapse of the anterior and posterior compartment of the vaginal is expected to be either totally or partially relieved. In one particular method, the vaginal cuff is secured to the sacrospinous ligament using two surgical anchors that are inserted via a vaginal route (incisionless) and placed through the vaginal cuff and into the sacrospinous ligament, with trailing sutures tied together within the vagina to approximate the vaginal cuff to the sacrospinous ligament. This method is described in U.S. Pat. No. 6,981,983, which is incorporated herein by reference in its entirety, and devices uniquely suited for this procedure are described in detail in co-pending patent application entitled “Anchor Tip Orientation Device and Method,” filed on Jun. 10, 2011 (application Ser. No. ______), which is also incorporated herein by reference in its entirety.

Although the previously described method is an improvement over prior known methods because it is an incisionless procedure, it has associated challenges since it does not allow for direct visualization or identification of nearby tissue structures. One particular problem encountered is bowel entrapment, where the nearby bowel is caught between the vaginal cuff and sacrospinous ligament when the anchors are inserted, sometimes leading to perforation of the bowel.

Accordingly, what is needed is an improved method for performing incisionless pelvic organ prolapse repairs that reduces or eliminates tissue entrapment issues described above.

### SUMMARY OF THE INVENTION

A method for treating pelvic organ prolapse conditions via a vaginal approach is provided, including the steps of identifying the presumptive apex of the vagina, identifying the ischial spine, and pressing the presumptive apex of the vagina onto the ischial spine. While maintaining contact between the presumptive apex of the vagina and the sacrospinous ligament, the method further provides continuously sweeping the presumptive apex of the vagina in a dorso-medial direction from the ischial spine along the sacrospinous ligament to a first location approximately two centimeters from the ischial spine and attaching the presumptive apex to the sacrospinous ligament at the first location using a first tissue anchor.

According to one embodiment, the method further includes prior to the attaching step, verifying by tactile feel the absence of thickening or compressible tissue between the presumptive apex and sacrospinous ligament.

According to yet another embodiment, the attaching step may also include inserting a first tissue anchor through the vagina and sacrospinous ligament, where the tissue anchor has a filamentary element coupled thereto that remains within the vagina. Further steps include, while maintaining contact between the vagina and the sacrospinous ligament, performing a further sweep in a dorso-medial direction to a second location approximately one centimeter from the location of the first anchor, inserting a second tissue anchor at the second location through the vagina and sacrospinous ligament, the tissue anchor having a filamentary element coupled thereto that remains within the vagina, and securing the filamentary elements of the first and second anchors together to thereby approximate the vagina to the sacrospinous ligament.

The first and second anchors and/or the filamentary elements may be made of an absorbable, biocompatible material.

According to yet another embodiment, the method may further include the step of performing one or more stitches through vaginal tissue with one or both filamentary elements prior to securing said filamentary elements together.

Also provided is a method for approximating a vaginal cuff to a sacrospinous ligament, including the steps of identifying the ischial spine, using one or more fingers inserted into the vagina, pressing the vagina onto the ischial spine, while maintaining contact between the vagina and the sacrospinous ligament, continuously sweeping the vagina in a dorso-medial direction from the ischial spine along the sacrospinous ligament to a first location approximately two centimeters from the ischial spine, and while maintaining contact between the vagina and the sacrospinous ligament, attaching the vagina to the sacrospinous ligament at the first location.

The method may further include, following the attaching step, while maintaining contact between the vagina and the sacrospinous ligament, continuing to sweep the vagina in a dorso-medial direction along the sacrospinous ligament to a second location one or more centimeters away from the first location; and while continuing to maintain contact between the vaginal and sacrospinous ligament, attaching the vagina to the sacrospinous ligament at said second location.

In yet another embodiment, the first and second attaching steps further include obtaining first and second anchors each having filamentary elements coupled to and extending therefrom, inserting the first and second anchors respectively through the vagina and sacrospinous ligament so that ends of filamentary elements remain within the vagina, and tying the filamentary elements of the first and second anchors together to thereby approximate the vagina to the sacrospinous ligament.

The first and second anchors and/or filamentary elements may be made of an absorbable, biocompatible material.

In yet another embodiment, the method further includes performing one or more stitches through vaginal tissue with one or both filamentary elements prior to securing said filamentary elements together.

These and other objects, features and advantages of the present invention will be apparent from the following detailed description of illustrative embodiments thereof, which is to be read in connection with the accompanying drawings.

### BRIEF DESCRIPTION OF THE DRAWINGS

FIGS. 1-11 illustrates various steps in a method for treating pelvic organ prolapsed conditions according to the present invention;

FIG. 12 illustrates an insertion device that can be used in performing the method illustrated in FIGS. 1-11;

FIGS. 13a and 13b are side views of the insertion device of FIG. 12 in the retracted and extended positions respectively;

FIG. 14 is an enlarged, cross-sectional view of the distal end of the insertion device of FIG. 12;

FIG. 15 is an enlarged, perspective view of the distal end of the insertion device of FIG. 12;

FIG. 16 illustrates how the anchor is mounted on the drive pin within the insertion device of FIG. 12; and

FIG. 17 is a sectional view of FIG. 16.

### DETAILED DESCRIPTION

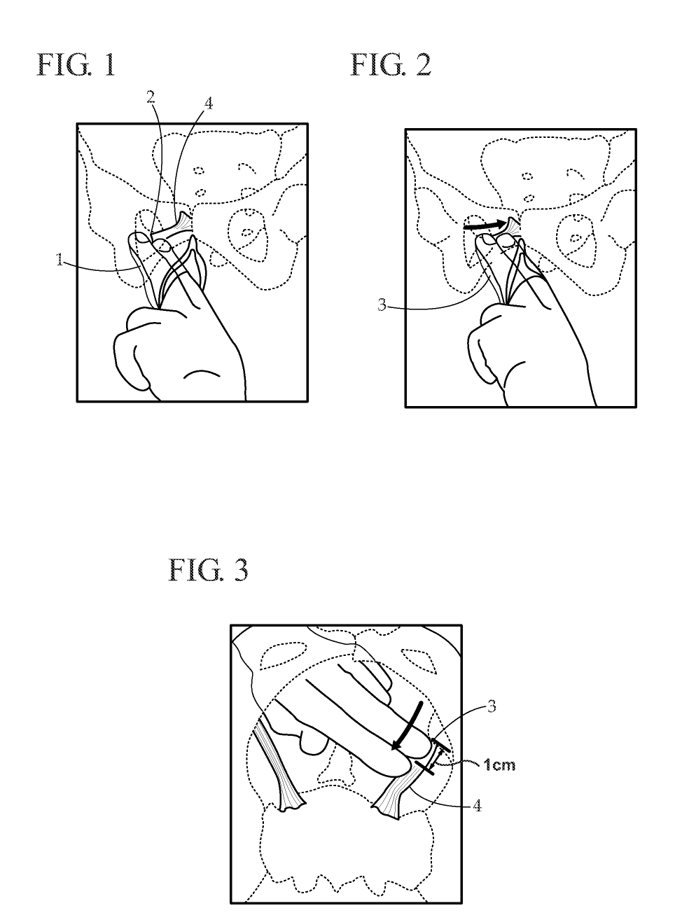

The improved method of the present invention will now be described in detail with reference to FIGS. 1-11.

Prior to initiating the procedure, a bowel preparation is performed to allow the bowel to be flexible enough to be moved or “swept” out of the deployment path of the tissue fastener or anchor. This is typically accomplished using an enema or the like. Similarly, the bladder should be drained to decrease its size and minimize the risk of interfering with the procedure.

The first step is for the surgeon to identify the site in the vagina where the presumptive apex of the vagina is located, which can be marked if desired. The term “presumptive apex” as used herein means the intended location at which the vagina will ultimately be fixed to the sacrospinous ligament. The surgeon will then insert the index and middle finger of the non-dominant hand into the vagina 1, and push the presumptive apex of the vagina up to the right spinous process or ischial spine 2, as is shown in FIG. 1. A dorso-medial sweeping motion is then conducted starting with the presumptive apex of the vagina pressed against the ischial spine 3 as shown in FIG. 3. The sweeping motion then continues in a dorso-medial direction away from the spinous process 2 until the finger tips overlie a point on the sacropsinous ligament approximately 2 centimeters medial to the ischial spine as shown in FIG. 3. During this sweeping motion the vagina must be kept in continuous contact with the sacrospinous ligament to prevent bowel entrapment. As the vagina is pressed against the sacrospinous ligament there should be no sense of increased thickening or compressible soft tissue between the fingers and the ligament, which would indicate the presence of unwanted tissue therebetween such as the bowel, rectum, bladder and/or peritoneum. If such thickening or compressed tissue is felt, the process should be repeated, starting with the identification of the starting point for the sweeping motion.

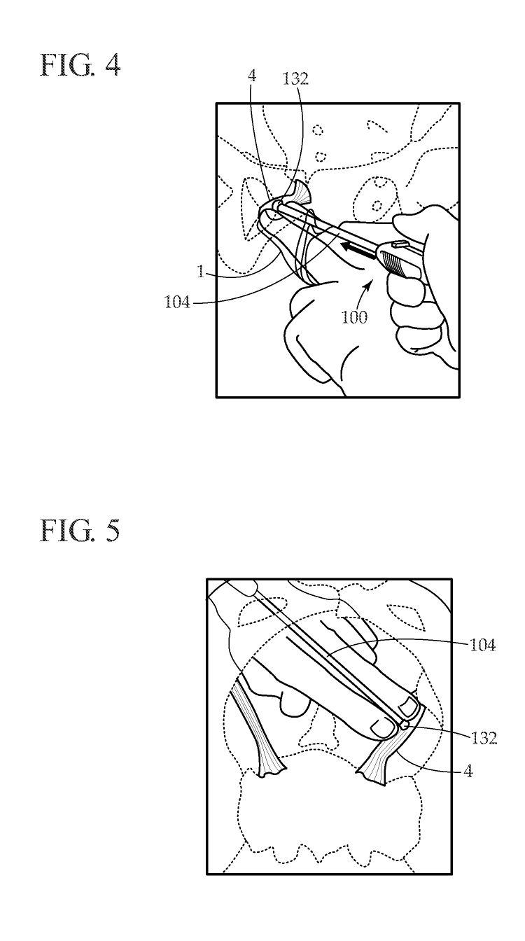

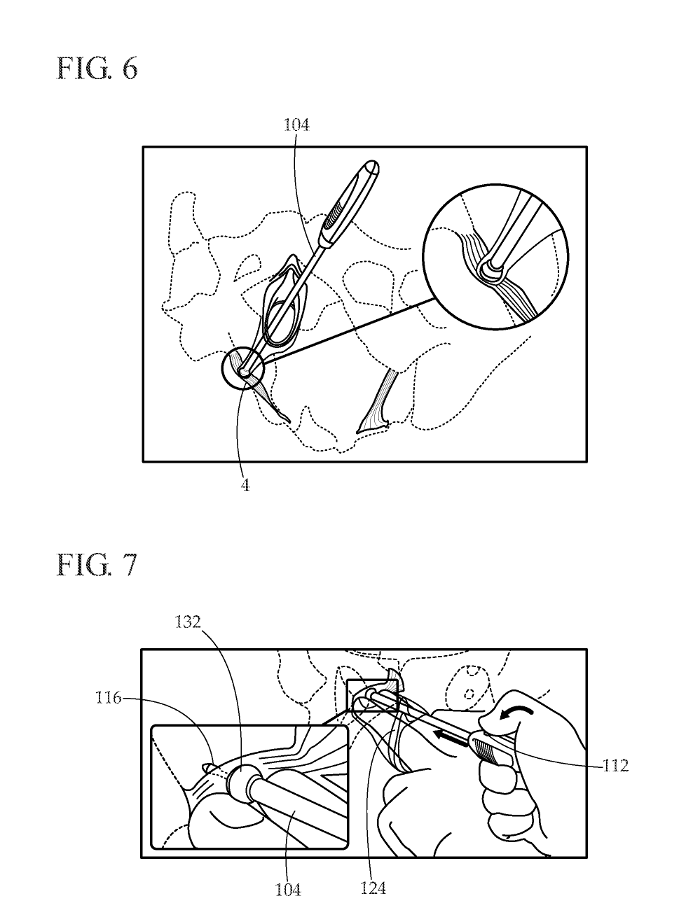

Once the sweep has been performed with no increased thickness detected and continuous contact has been maintained between the presumptive apex and the sacrospinous ligament, the insertion device 102 (described further below) can be inserted into the vagina. The outer barrel 104 of the device is slid along the dorsum of the two fingers until the outer barrel tip 132 is firmly in contact with the sacrospinous ligament 4 as shown in FIGS. 4 and 5. The axis of the outer barrel should be substantially perpendicular to the sacrospinous ligament (as shown in FIG. 6) to improve the likelihood that the anchor will penetrate the sacrospinous ligament properly. Once properly positioned, the actuator or release button 112 is pressed, allowing deployment of the anchor 116 through the vaginal wall and sacrospinous ligament as shown in FIG. 7. The insertion device is then withdrawn, leaving the anchor in place and the filamentary element 124 dangling within the vagina.

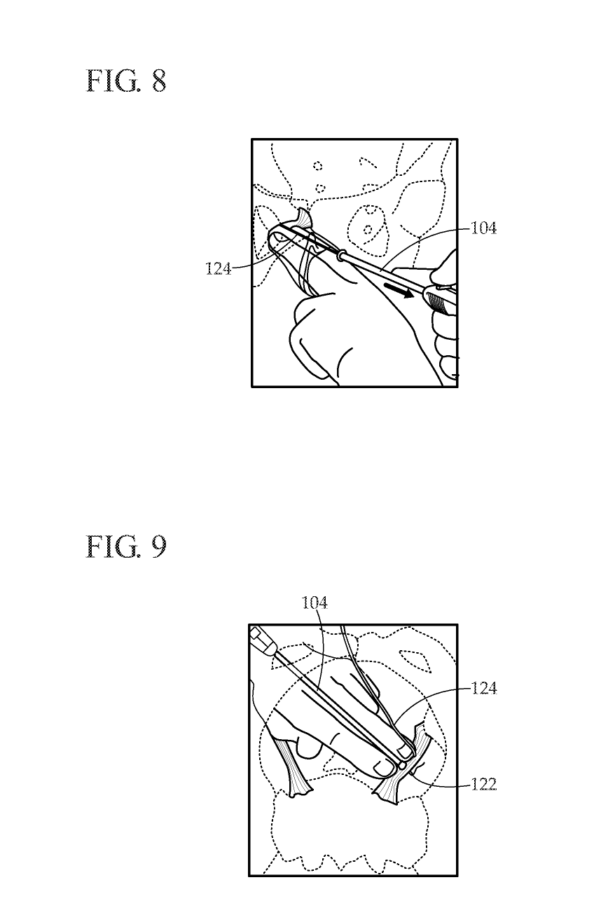

While still maintaining pressure on the sacrospinous ligament, a further dorso-medial sweep is performed another 1 centimeter from the first anchor, then the second inserter device and anchor is inserted into the vagina and the anchor deployed in the same manner as described above as shown in FIGS. 8 and 9.

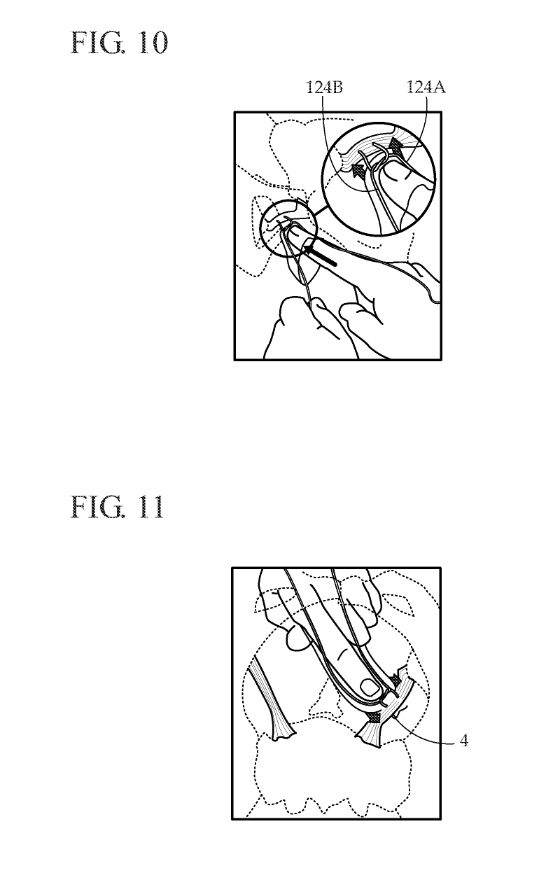

Following deployment of the second anchor and removal of the second insertion device, a rectal exam is performed to ensure that no rectal penetration has occurred. The two suture tails 124a, 124b are then tied together to approximate the vaginal wall to the sacrospinous ligament as shown in FIGS. 10 and 11. Prior to tying the suture tails together, one or more additional stitches may be taken through tissue with one or both of the suture tails to separate the tails and to put more vaginal tissue in contact with the sacrospinous ligament. The suture tails are then trimmed, and the level of apical support achieved is determined.

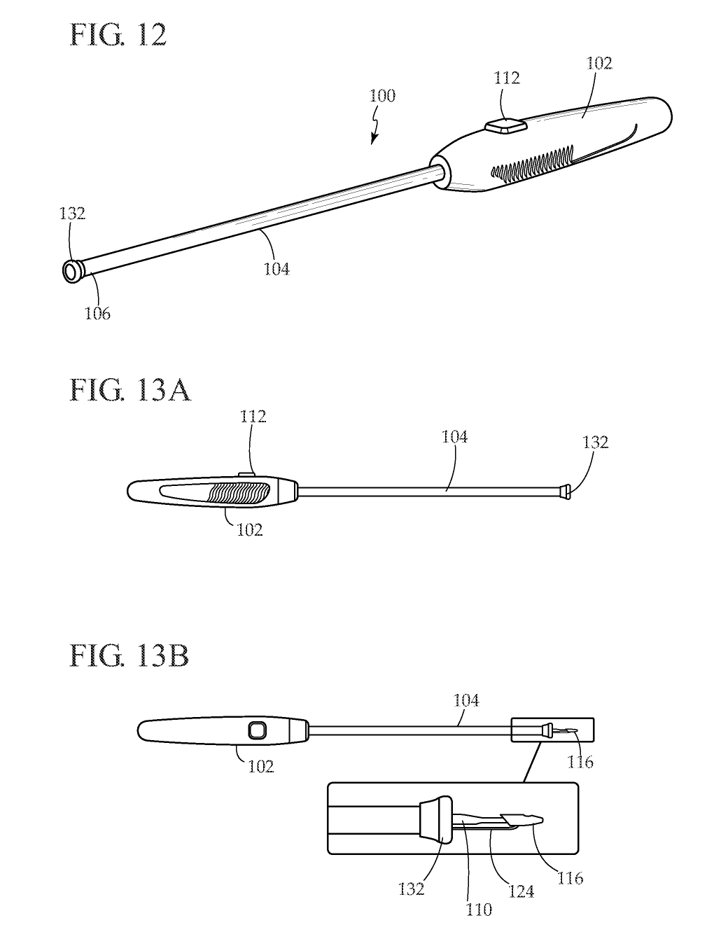

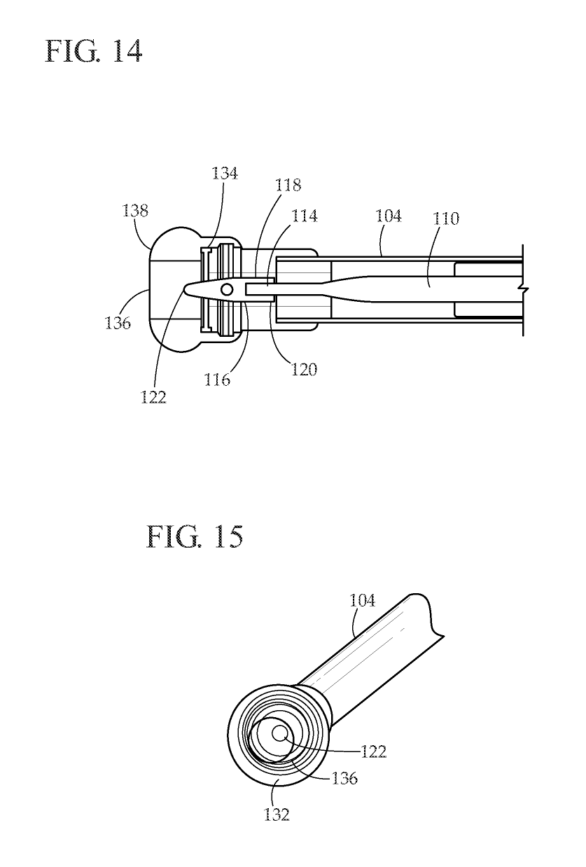

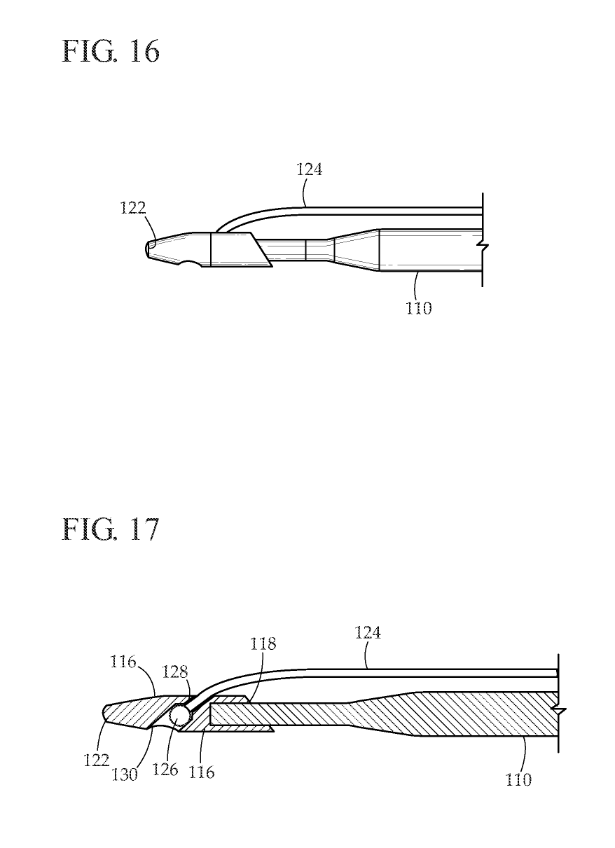

The insertion device and anchor are illustrated in greater detail in FIGS. 12-17. The insertion device 100 includes a handle 102, and an outer barrel 104 extending outwardly therefrom to a distal end 106. As is best seen in FIG. 14, positioned within the outer barrel 104 is an elongated drive pin 110 with an implantable surgical anchor 116 mounted on its distal end 114. As will be described in further detail below, the outer barrel 104 is movable relative to the drive pin 110 by actuation of button 112 between a first position where the anchor is covered by the outer barrel and outer barrel tip 132 as shown in FIG. 16a, and a second position where the anchor extends distally beyond, and is not covered by, the outer barrel and outer barrel tip. A channel 118 extending inwardly from the proximal end 120 of the anchor is configured to fit over the distal end 114 of the drive pin as shown in FIGS. 14 and 16-17. The channel 118 and distal end 114 of the drive pin are sized and shaped so as to form an interference fit. The distal end 122 of the anchor is tapered, preferably sufficiently to form a tissue penetrating distal end. In the illustrated embodiment, a filamentary element 124 including a distal, bulbous end 126 also is coupled to and extends from the anchor. The filamentary element 124 passes through a second channel 128 extending laterally through the anchor, with the bulbous end of the filamentary element being seated within a larger portion 130 of the channel as shown in FIG. 17 to thereby prevent the filamentary element from being pulled free from the anchor.

In a preferred embodiment, the anchor is comprised of an absorbable, biocompatible material such as polydioxanone. The anchor is approximately 0.299-0.385 inches in length with a maximum outer diameter of approximately 0.080-0.100 inches, and the channel 118 is approximately 0.035 inches in diameter and 0.099-0.137 inches in length. The distal end of the corresponding drive pin preferably has an outer diameter of approximately 0.035+/−0.005 inches so as to form an interference fit, but not so tight as to impede release of the anchor from the drive pin during implantation. The filamentary element 124 is preferably a size 2-0 or 0 suture comprised of an absorbable, biocompatible material such as polydioxanone, or any other suitable suture material.

Maintaining the positioning of the anchor 116 relative to the drive pin 110 can be critical when trying to implant the anchors. Often the device must be manipulated within the body to ensure implantation of the anchor in the right location. Further, the filamentary element 124 that extends from the anchor remains under slight tension. The frictional fit between the anchor and the distal end of the drive bore is insufficient to prevent the anchor from loosening or becoming slightly askew during manipulation of the delivery device. When out of position, the anchor will not drive straight into tissue as intended when deployed. In some cases, the anchor will turn and jam or break during deployment. Further, if the frictional engagement is too great, deployment of the anchor may become difficult if not impossible.

In order to further stabilize positioning of the anchor prior to implantation, the insertion device further includes an outer barrel tip 132 at the distal end of the insertion device. The outer barrel tip extends circumferentially around the distal end 106 of the outer barrel 104, and surrounds and extends distally of the distal tip 122 of the anchor when the insertion device is in first position shown in FIG. 13a. The outer barrel tip includes within it an elastomeric septum 134, which engages the tip of the anchor circumferentially as shown in FIG. 14. The outer barrel tip 132 also includes an opening or channel 136 extending inwardly from the distal end 138 so that the distal tip of the anchor is visible from the distal end of the insertion device as shown in FIG. 15. The outer barrel tip may also be comprised of a clear or translucent material so that the anchor position within the tip is readily visible by a user. During deployment, the outer barrel 104 and coupled outer barrel tip 132 is retracted relative to the drive pin 110 and anchor 116 by activating button 112 via any well known means. As this occurs, the anchor drives through the elastomeric septum 132 in the outer barrel tip in a distal direction until the drive pin and anchor are exposed as illustrated in FIG. 13b.

Although illustrative embodiments of the present invention have been described herein with reference to the accompanying drawings, it is to be understood that the invention is not limited to those precise embodiments and that various other changes and modifications may be effected herein by one skilled in the art without departing from the scope or spirit of the invention.

What is claimed is:

1. A method for treating pelvic organ prolapse conditions via a vaginal approach, comprising:
identifying the presumptive apex of the vagina;
identifying the ischial spine;
pressing the presumptive apex of the vagina onto the ischial spine;
while maintaining contact between the presumptive apex of the vagina and the sacrospinous ligament, continuously sweeping the presumptive apex of the vagina in a dorso-medial direction from the ischial spine along the sacrospinous ligament to a first location approximately two centimeters from the ischial spine;
while maintaining contact between the presumptive apex of the vagina and the sacrospinous ligament, attaching the presumptive apex to the sacrospinous ligament at the first location using a first tissue anchor.

2. The method according to claim 1, further comprising, prior to the attaching step, verifying by tactile feel the absence of thickening or compressible tissue between the presumptive apex and sacrospinous ligament.

3. The method according to claim 1, wherein the attaching step further comprises, inserting a first tissue anchor through the vagina and sacrospinous ligament, the tissue anchor having a filamentary element coupled thereto that remains within the vagina;
while maintaining contact between the vagina and the sacrospinous ligament, performing a further sweep in a dorso-medial direction to a second location approximately one centimeter from the location of the first anchor; and
inserting a second tissue anchor at the second location through the vagina and sacrospinous ligament, the tissue anchor having a filamentary element coupled thereto that remains within the vagina; and
securing the filamentary elements of the first and second anchors together to thereby approximate the vagina to the sacrospinous ligament.

4. The method according to claim 3, wherein the first and second anchors are comprised of an absorbable, biocompatible material.

5. The method according to claim 4, wherein the filamentary elements of the first and second anchors are comprised of an absorbable, biocompatible material.

6. The method according to claim 3, further comprising performing one or more stitches through vaginal tissue with one or both filamentary elements prior to securing said filamentary elements together.

7. A method for approximating a vaginal cuff to a sacrospinous ligament, comprising:
identifying the ischial spine;
using one or more fingers inserted into the vagina, pressing the vagina onto the ischial spine;
while maintaining contact between the vagina and the sacrospinous ligament, continuously sweeping the vagina in a dorso-medial direction from the ischial spine along the sacrospinous ligament to a first location approximately two centimeters from the ischial spine;
while maintaining contact between the vagina and the sacrospinous ligament, attaching the vagina to the sacrospinous ligament at the first location.

8. The method according to claim 7, further comprising the steps of, following the attaching step, while maintaining contact between the vagina and the sacrospinous ligament, continuing to sweep the vagina in a dorso-medial direction along the sacrospinous ligament to a second location one or more centimeters away from the first location; and while continuing to maintain contact between the vaginal and sacrospinous ligament, attaching the vagina to the sacrospinous ligament at said second location.

9. The method according to claim 8, wherein the first and second attaching steps further comprise obtaining first and second anchors each having filamentary elements coupled to and extending therefrom; and inserting the first and second anchors respectively through the vagina and sacrospinous ligament so that ends of filamentary elements remain within the vagina; and tying the filamentary elements of the first and second anchors together to thereby approximate the vagina to the sacrospinous ligament.

10. The method according to claim 8, wherein the first and second anchors are comprised of an absorbable, biocompatible material.

11. The method according to claim 10, wherein the filamentary elements of the first and second anchors are comprised of an absorbable, biocompatible material.

12. The method according to claim 8, further comprising performing one or more stitches through vaginal tissue with one or both filamentary elements prior to securing said filamentary elements together.
